(19) United States
(12) Patent Application Publication
(10) Pub. No.: US 2016/0091474 A1
(43) Pub. Date: Mar. 31, 2016

(54) Method and a System for Determining at Least One Forecasted Air Quality Health Effect Caused in a Determined Geographical Area by at Least One Air Pollutant

(71) Applicant: Tanguy Griffon, Geneva (CH)

(72) Inventor: Tanguy Griffon, Geneva (CH)

(21) Appl. No.: 14/499,770

(22) Filed: Sep. 29, 2014

(51) Int. Cl. G01N 33/00; G01W 1/10

(52) U.S. Cl. CPC G01N 33/0036; G01W 1/10

(57) ABSTRACT

A method for hourly, daily, weekly, monthly, quarterly and annual forecasted air quality health effects caused by air pollutants generated over a determined geographical area and a system implementing the method.

### BACKGROUND OF THE INVENTION

1. Field of the Invention

The invention relates to processes for determining forecasted air quality health effects caused by air pollutants. The invention relates in particular to a method and a system for determining at least one forecasted air quality health effect caused in a determined geographical area by at least one air pollutant.

2. Incorporation by Reference

All patents, patent applications, documents and references mentioned herein are incorporated herein by reference and may be employed in the practice of the invention.

3. Description of the Prior Art

Air pollution can be described as a contamination of the atmosphere by gaseous, liquid or solid wastes or by-products that can endanger human health and welfare of plants and animals, attack materials, reduce visibility, or produce undesirable odors. Greenhouse gases are air pollutants given their damaging effects on public health, welfare and the environment. An air pollutant can be defined on the concentration of chemicals present in the environment. If the concentration of any chemical is above the concentration of the chemical in the air, then it is termed as an air pollutant. There are two basic physical forms of air pollutants. The first one is the gaseous form and the second one, are particles such as smoke, dust, mists, and fly ash. Primary pollutants remain in the same chemical form as they are released from a source directly into the atmosphere and secondary pollutants are a result of chemical reaction between two or more pollutants. Over one hundred air pollutants have been identified including halogen compounds such as fluorine (F), chlorine (Cl), bromine (Br), and iodine (I), nitrogen compounds such as Nitrogen Dioxide (NO2), oxygen compounds such as Ozone (O3), sulfur compounds such as Sulfur Dioxide (SO2), Carbon monoxide (CO), Volatile Organic Compounds (VOCs), Particulate matter (PM) as presented in the process for PM2.5, Persistent free radicals, Chlorofluorocarbons (CFCs), Ammonia (NH3), Radioactive pollutants and Persistent organic pollutants (POPs). As some pollutants are released by natural sources like volcanoes, coniferous forests, and hot springs, the effect of this pollution is small compared to that of anthropogenic sources such as industrial sources, power generation, heat generation, waste disposal and the operation of internal-combustion engines.

Outdoor air pollution leads to major adverse health effects and has been declared as a leading environmental health risk by the World Health Organization. Health effects include premature deaths, asthma attacks, heart attacks, strokes, cardiovascular harm, lung cancers, low birth weight, infant mortality, wheezing, coughing, shortness of breath, susceptibility of infection, lung tissue redness and swelling. People such as individuals with underlying respiratory and cardiovascular diseases, children, elderly and pregnant women are more prone to develop harmful health effects when exposed to air pollutants.

According to epidemiological studies, fine Particulate Matter PM2.5 is usually the reference air pollutant believed to pose the greatest health risks. Particulate matter is the term for particles found in the air less than 2.5 micrometers in diameter and because of their small size, can lodge deeply into the lungs.

As of today, there is no method for precisely determining health effects because air quality information is provided at a coarse resolution and with some uncertainty. Indeed, air pollution information and maps are mainly provided to users under the form of an Air Quality Index (AQI) by websites, weather channels, mobile apps or locally by sensors, which do not allow individuals to view their real-time personal exposures, to find clean air areas around them as well as to visualize future forecasts at a high resolution. An example of AQI is shown in FIG. 9. It indicates how clean or polluted the air is, and what associated health effects might be of concern for individuals. It focuses on health effects that may be experienced within a few hours or days after breathing polluted air.

Unfortunately, these approaches are not usable on a personal level due to their coarse temporal resolution (daily), spatial resolution (state or county level), uncertainty and limited future forecasts. These methods require the insertion of high resolution and accurate air quality data and despite their constant improvements; concentrations are estimated by extrapolating concentrations of a single gas over large areas with data from a sparse monitoring network. These linear approaches do not consider the high spatial and temporal variability due to pollutants, weather and climate interactions, which leads to uncertainties in air pollutant concentrations and health effects.

In addition, air quality information is also provided by free apps available on the market. They provide sparse monitoring stations data (O3, PM) from ground monitors, which are not usable on a personal level. Portable sensors combined with mobile apps include small devices to engage users in personal monitoring. A user needs to purchase an extra sensor and wear it continuously, which is an impediment and which does not provide a view on local areas around a user or future forecasts. Sensor networks are small fixed low-cost devices located outside. Those sensors provide readings in one location without tracking personal exposures and local areas around a user or providing future forecasts.

In the same perspective, as of today, the attributable public health burden on a population from an air pollutant can only be estimated from historical data on an ad-hoc basis and with some uncertainty related to exposure assessment. Current methods to estimate health effects are software based. A reference in the field is the U.S. EPA Environmental Benefits Mapping and Analysis Program (BenMAP) http://www.epa.gov/air/benmap/, which estimates the health impacts and economic benefits occurring when populations experience changes in air quality. It currently uses ground monitoring data or air quality forecasts at a county or 12×12 km² (e.g. 7.46×7.46 sq. miles) resolution to provide health effects at a county or 12×12 km² resolution up to the year 2008. These software determine health effects from health impact functions reported in the epidemiological literature. They quantify the relationship between changes in air pollution and adverse health impacts with the following components:
Effect estimate (β or “beta”), which quantifies the change in health effects per unit of change in a pollutant and is derived from an epidemiological study.
Change in exposure (ΔPM), which estimates the change in the concentration of ambient PM2.5
Baseline incidence rate (yₒ), which is the incidence rate before the change in PM2.5.
Control incidence rate (y), which is the health effect incidence rate after the change in PM2.5.
Population (pop), which is the affected population from the epidemiological study.
Health effects (Δhealth), which are the resulting health effects from the change in air pollutants.

The three main forms of health impact assessment are the linear, log-linear and logistic forms as published in BenMAP. A functional form is chosen by the researcher, and the function parameters are estimated using pollutant data (e.g., daily levels of PM2.5) and the health response (e.g., asthma exacerbations, cough) as shown in FIG. 7. A linear relationship expresses the change in the rate of the adverse health effect from the baseline rate (y₀) to the rate after control (y_{c}) associated with a change from PM₀ to PM_{c} as follows:

ΔHealth=Δy·pop=(yₒ−y_{c})·pop=β·(PMₒ−PM_{c})=β·ΔPM·pop

A typical log-linear health impact function defines the relationship between ΔPM and Δy as follows:

$$Δ Health = Δ y \cdot pop = ( 1 - \frac{1}{exp ( β . Δ PM )} ) \cdot {y}_{o} \cdot pop$$

A logistic health impact function defines the relationship between ΔPM and Δy as follows:

$$Δ Health = Δ y \cdot pop = ( 1 - \frac{1}{( 1 - {y}_{o} ) {e}^{β Δ PM} + {y}_{o}} ) \cdot {y}_{o} \cdot pop$$

A clear challenge of this health impact assessment is exposure assessment. The granularity and reliability of air quality modeling is critical to precisely assess health effects. Air pollution data is based on software generated air quality maps or ground monitoring data where observations are available. These methods require the insertion of high resolution and accurate air quality data and despite their constant improvement, outdoor exposures are also estimated by extrapolating concentrations of a single gas over large areas with historical data from a sparse monitoring network. In the same perspective as the Air Quality Index methods, these linear approaches do not consider the high spatial and temporal variability due to pollutants, weather and climate interactions.

Hence, as of today, there is no method that would enable individuals to prevent accurately their health effects from air pollution depending on their pathology in any location in near real-time as well as in the future.

Therefore, as of today, there are no system that individuals can use to predict with accuracy and when and where local air quality will be more or less healthy in near real-time and in the future.

There is also no system to automatically alert a user exposed to harmful levels and to enable him or her to find clean air around, thus preventing health effects.

A measurement that is continuous, accurate and in-situ of air pollutants taking into account the high spatial and temporal variability due to multiple pollutants, weather and climate interactions assessing on a planetary scale up to local areas effects on individuals will improve assessment of hourly concentrations forecasts. Integrating especially continuous physical observations such as satellite and ground observations into the air quality forecasts will ensure a higher reliability and spatial coverage. This top-down atmospheric measurement process can complement current approaches by forecasting hourly concentrations and health effects on a continuous basis currently and in the future. This will enable the general public to prevent exposures and individuals with specific pathologies to better manage their disease as well as for countries and entities to continuously improve the health prevention efficiency over time. This will support health effects and costs reduction and help solve this major public health challenge.

### SUMMARY OF THE INVENTION

A first purpose of the invention is to provide an improved method for determining at least one forecasted air quality health effect by geographical area, compared to current methods available, which can be used to prevent with accuracy, timeliness and in the future the health effects of air pollutants.

In other terms, in the technical field of determining health effects induced by air pollutants in geographical areas, the method according to the invention allows a determination of health effects of air pollutants more accurately, timely and in the future than current methods, in particular more accurately, timely and forecasted than BenMAP.

In particular, a first advantage of the method according to the invention is to resolve smaller spatial scales in near real time to obtain the concentrations of an air pollutant and the Air Quality Index (AQI) in the current hour and with hourly and daily forecasts with a resolution of 3×3 km2 (1.86×1.86 sq. miles) over an entire country or continental region, which has never been done to current knowledge. With the determination of hourly concentrations forecasts and AQI, one advantage of the method according to the invention is to determine, for areas that can be between 1 km2 and 10,000 km2 (e.g. 0.3861 sq. miles and 3861 sq. miles), the hourly concentrations and AQI forecasts with an accuracy above 5%. In other terms, the first advantage of the method and of the system according to the invention is to provide a more accurate forecast of air pollutants and AQI, enabling one to determine health effects caused by air pollutants, from a global scale up to an area whose surface is 1×1 km2.

Another goal of the invention is to take into account health effects such as asthma, acute bronchitis, acute myocardial infarction, cardiovascular, respiratory, chronic bronchitis, chronic lung, congestive heart failure, cough, dysrhythmia, ischemic heart, lower respiratory symptoms, mortality from all cause, mortality from ischemic heart disease, mortality from lung cancer, pneumonia, shortness of breath, upper respiratory symptoms, wheeze and for any new disease, which can be determined from a research indicating the incidence of a disease from an air pollutant.

In particular, a second characteristic of the invention is to resolve smaller spatial scales to obtain the averaged values of PM2.5 concentrations on an hourly, daily, weekly, monthly, quarterly and annual basis as well as the health effects, notably of Asthma, in the number of events hourly, daily, weekly, monthly, quarterly or annually with a resolution of 3×3 km2 in the current and future year e.g. 2014, 2015, etc, which has never been done to current knowledge. With the determination of health effects, one purpose of the process according to the invention is therefore to determine, for areas that can be between 1 km2 and 10,000 km2, the number of health events with an accuracy above 5%. With the integration of multiple observations in real-time and by taking into account the multiple interactions of weather, climate and air pollutants, this near real-time and forecasted, uniform and global measurement of air pollutants has advantages of determining homogenously over an entire continental region or nation the health effects and reducing the uncertainty compared to current sparse monitoring networks or computer modeled air quality.

A second goal of the invention is to provide a system for forecasting health effects caused by air pollutants which can be combined, notably with a wearable device such as a mobile application to geolocalize and automatically alert an individual when exposed to harmful air pollutant levels and to allow one to view hourly air pollutants in near real time in one personal location as well as to visualize and find locations with lower levels of air pollution around to prevent exposures. It will also allow a user to visualize over days and weeks the evolution of this air pollution at a high resolution. This information can be provided in the form of air pollutant concentrations or Air Quality Index to allow the individual to assess potential near real time and future health effects and to avoid harmful locations by changing behavior on the short term such as by changing location, staying indoor, using preventive medicine or planning activities ahead. Moreover, when an individual is equipped with a wearable device and indicates his or her pathology such as a respiratory or a cardiovascular health problem, the individual can be geolocalized and automatically alerted of the high incidence of this health effect when located in a harmful location. The individual can then visualize and find locations with lower levels of his or her pathology in the vicinity in the past and in the future, over hours, days, weeks and months.

An advantage of the system according to the invention is therefore to provide a system with an Application Programming Interface (API) that can directly be interfaced with the prevention management system of an entity to determine preventive activities to undertake such as relocating individuals or providing preventive medicine or best practices in order to limit the number of health effects or to automatize prevention actions such as alerting, calling or emailing individuals at risk to help reduce exposures.

Specific hardware and software means implementing the method according to the invention and ensuring interfacing with the wearable device or application programming interface are installed within the wearable device of an individual or within an entity depending on their activity, the processes implemented by them and the type of health effect. Specific hardware and software means implementing the method according to the invention and ensuring interfacing with a wearable device can be downloaded on the device for personal usage or interfaced with the API in the prevention management system of an entity. The method according to the invention can then be used to avoid local air pollution and set up preventive actions to reduce the number of future health events in local areas, counties, cities, states, depending on the levels and the types of health effects measured (ex: asthma attacks close to refineries or highways). This enables one to obtain an automated implementation of health effects prevention and to progressively control its effectiveness by assessing health events and costs reduction.

These goals are reached and these advantages are provided by, according to a first aspect of the invention, a method for determining at least one forecasted air quality health effect caused in a determined geographical area by at least one air pollutant, said method being performed by a system comprising at least one computing device, which comprises one or more processors and one or more computer-readable storage media operatively coupled to at least one of the processors, wherein said method includes:
a step of retrieving, by means of an air pollutant measurement module, hourly concentrations forecasts of said at least one air pollutant;
a step of determining, by means of an exposure module, population exposure forecasts using said hourly concentrations forecasts and population related data;
a step of deriving, by means of an incidence module, said at least one forecasted air quality health effect caused by said air pollutant using said population exposure forecasts;
a step of localizing, by means of a first geocoding module comprising at least one geographic information system, said at least one forecasted air quality health effect caused by said air pollutant so as to assess said at least one forecasted air quality health effect in said determined geographical area.

According to one characteristic, the surface of said determined geographical area may be comprised between 1 km2 and 10,000 km2.

According to another characteristic, said at least one health effect may be selected from the group consisting of asthma, acute bronchitis, acute myocardial infarction, cardiovascular, respiratory, chronic bronchitis, chronic lung, congestive heart failure, cough, dysrhythmia, ischemic heart, lower respiratory symptoms, mortality from all cause, mortality from ischemic heart disease, mortality from lung cancer, pneumonia, shortness of breath, upper respiratory symptoms and wheeze.

According to another characteristic, said step of retrieving hourly concentrations forecasts of said at least one air pollutant may comprise the following steps:
use hourly concentration measurements of said at least one air pollutant measured in a first plurality of locations distributed on the entire terrestrial globe and save said hourly concentration measurements in an observation module;
use hourly flux measurements of said at least one air pollutant measured in a second plurality of locations distributed on the entire globe and save said hourly flux measurements in said observation module;
use measurements of satellite parameters, meteorological parameters, marine parameters and ecosystem parameters measured in a third plurality of locations distributed on the terrestrial globe and save said satellite parameters, said meteorological parameters, said marine parameters and said ecosystem parameters in said observation module;
extract, by means of an extraction module, weather forecast data from at least one data source;
perform a flux evolution modeling of said at least one air pollutant on the globe by means of an exchange module modeling the natural and anthropogenic sources and sinks;
perform an hourly anthropogenic emissions modeling of said at least one air pollutant by means of an ascending inventories module, said ascending inventories module integrating the raw data of emissions for a plurality of facilities;
perform, using said flux evolution modeling, said hourly anthropogenic emissions modeling and said weather forecast data, an atmospheric transport forecast of said air pollutant by means of a transport module;
calculate final fluxes of said at least one air pollutant, by means of a data inversion and assimilation module, by making use of said flux evolution modeling, said hourly anthropogenic emissions modeling, said atmospheric transport forecast and said measurements saved in said observation module;
weight, by means of a weighting module, said final fluxes so as to provide final weighted fluxes;
calculate, using said final weighted fluxes and said hourly anthropogenic emissions modeling, the hourly emissions of said at least one air pollutant of said geographical area, by means of a second geocoding module comprising at least one geographic information system; and
adjust said raw data of emissions for a plurality of facilities by making use of said hourly emissions of said at least one air pollutant of said geographical area; and
retrieve said hourly concentrations forecasts of said at least one air pollutant from the said atmospheric transport forecast.

According to another characteristic, said transport module may make use of the RAP-Chem model and the HRRR-Chem model.

According to another characteristic, said population related data may include a block-level census population data, a population grid, a county level forecast and a population forecast.

According to another characteristic, said incidence module may use health incidence data, concentration response functions and concentration response parameters from epidemiological studies and medical research used by BenMAP as well as the Air Quality Index issued by U.S. EPA.

According to the invention, a system for determining at least one forecasted air quality health effect caused in a determined geographical area by at least one air pollutant, said system comprising at least one computing device which comprises one or more processors, one or more computer-readable storage media operatively coupled to at least one of the processors, and implementing an air pollutant measurement module, an exposure module, an incidence module and at least one geocoding module comprising at least one geographic information system, performs a method for determining at least one forecasted air quality health effect caused in a determined geographical area by at least one air pollutant, said method including the following steps
a step of retrieving hourly concentrations forecasts of said at least one air pollutant, wherein said step of retrieving is performed by means of said air pollutant measurement module;
a step of determining population exposure forecasts of said at least one air pollutant using said hourly concentrations forecasts and population related data, wherein said step of determining is performed by means of said exposure module;
a step of deriving said at least one forecasted air quality health effect caused by said air pollutant using said population exposure forecasts, wherein said step of deriving is performed by means of said incidence module; and
a step of localizing said at least one forecasted air quality health effect caused by said air pollutant so as to assess said at least one forecasted air quality health effect in said determined geographical area, wherein said step of localizing is performed by means of said geocoding module.

According to one characteristic, said at least one forecasted air quality health effect may be determined on an hourly basis.

According to another characteristic, said at least one forecasted air quality health effect may be determined on a daily basis.

According to another characteristic, wherein said at least one forecasted air quality health effect may be determined on a weekly basis.

According to another characteristic, said at least one forecasted air quality health effect may be determined on a monthly basis.

According to another characteristic, wherein said at least one forecasted air quality health effect may be determined on a quarterly basis.

According to another characteristic, wherein said at least one forecasted air quality health effect may be determined on an annual basis.

According to another characteristic, said system may further include an interfacing module for interfacing with at least one software application executed by a wearable device.

According to another characteristic, wherein said wearable device may be a smartphone.

According to another characteristic, said system may further comprise an online platform comprising at least one server hosting at least one website.

According to another characteristic, said system may further comprise a framework allowing said system to be linked to a remote sanitary prevention management system.

It is appropriate to establish that in the meaning of the present invention and throughout the description, the word “module” must be interpreted in the computer science sense of the term. Indeed, all modules of the process according to the present invention are implemented in the form of software, hardware or a combination of both. Each module of the method can advantageously, depending on its role, be implemented using computer equipment means, notably means of calculation (computers, dedicated servers, mainframes, etc), communication systems (WAN, LAN), but also software, notably database management systems, modeling software, calculation software etc. The method can also be implemented in the form of a single software package, possibly accessible online via the Internet.

### BRIEF DESCRIPTION OF THE DRAWINGS

The invention will be better understood by a person skilled in the art thanks to the detailed description of different embodiments in relation with the accompanying drawings, in which:

FIG. 1 is a block diagram illustrating the steps and components of the method;

FIG. 2 is a table illustrating the primary and secondary PM2.5 particles sources and sinks;

FIG. 3 is a table illustrating the Exposure Module Variables and Block-Level Census Variables;

FIG. 4 is a table illustrating the MARS assignment;

FIG. 5 is a table illustrating the Population grid-cell example;

FIG. 6 is a table illustrating the Population grid-cell weight example;

FIG. 7 is a table illustrating the PM2.5 health effects from epidemiological studies;

FIG. 8 is a table illustrating the asthma incidence and prevalence rates;

FIG. 9 is a table illustrating the U.S. EPA conversion between PM2.5, AQI and health effects;

FIG. 10 presents a block diagram illustrating a system according to the invention;

FIG. 11 shows the wearable device such as mobile application or smart watch with automatic alerts from hourly AQI and air pollutant concentrations;

FIG. 12 shows the health prevention on a wearable device such as a mobile app with automatic alerts from averaged forecasted air quality health effects;

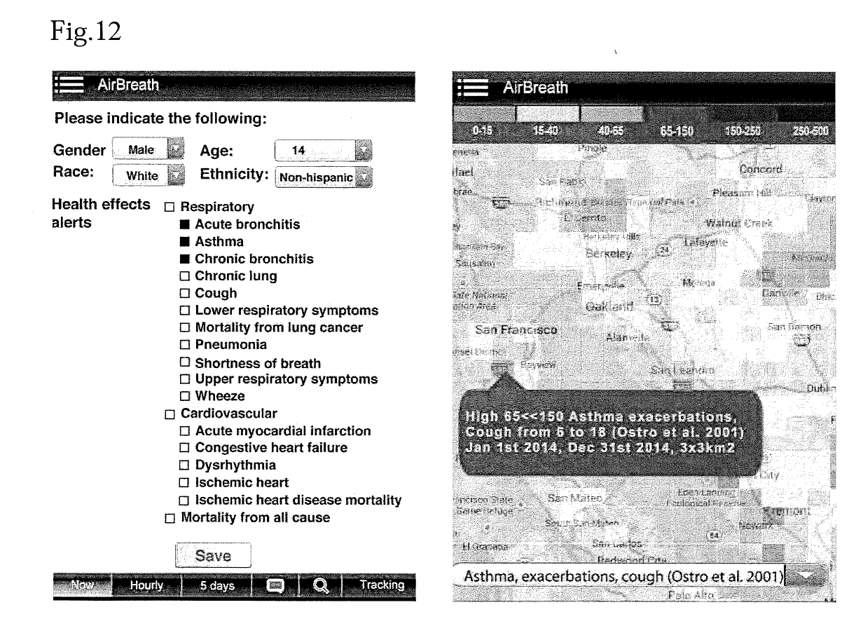

FIG. 13 shows the health prevention platform;

FIG. 14 shows the health prevention application programming interface.

### DETAILED DESCRIPTION OF THE PREFERRED EMBODIMENTS

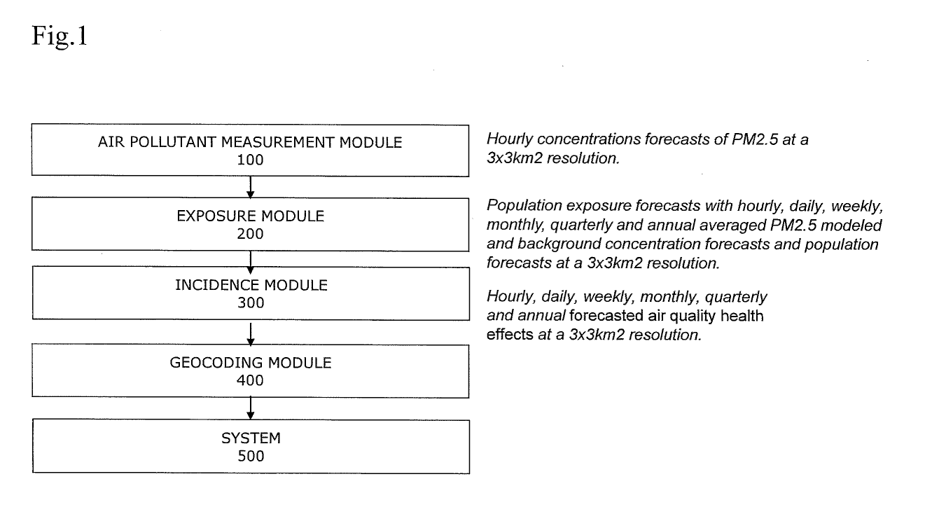

FIG. 1 presents, in a general manner, the different steps of the method according to the invention. The invention concerns a method and an accurate system to prevent health effects of an air pollutant in a determined geographical area, in particular in an area of which the surface is between 1 km2 and 10,000 km2. The method is initially presented for asthma and fine particles PM2.5 on the United States and the same process is used for the other health effects, air pollutants and geographies.

I. Asthma

1. Air Pollutant Measurement Module

Since greenhouse gases are air pollutants, the method described in US 2013/01790178 A1 is applied to the air pollutant measurement module (block 100) to accurately measure the hourly concentrations of fine particles PM2.5 on a predetermined geographical area from 1 km2 to 10,000 km2. For any other pollutants, the same method is used. These pollutants preferably include halogen compounds such as fluorine (F), chlorine (Cl), bromine (Br), and iodine (I), nitrogen compounds such as Nitrogen Dioxide (NO2), oxygen compounds such as Ozone (O3), sulfur compounds such as Sulfur Dioxide (SO2), Carbon monoxide (CO), Volatile Organic Compounds (VOCs), Other Particulate matters (PM), Persistent free radicals, Chlorofluorocarbons (CFCs), Ammonia (NH3), Radioactive pollutants and Persistent organic pollutants (POPS). In particular, measurement of Sulfur dioxide (SO2), Ozone (O3), Nitrogen Oxides (NOx) and Carbon Monoxide (CO) are already detailed in the method described for greenhouse gas in US 2013/0179078 A1.

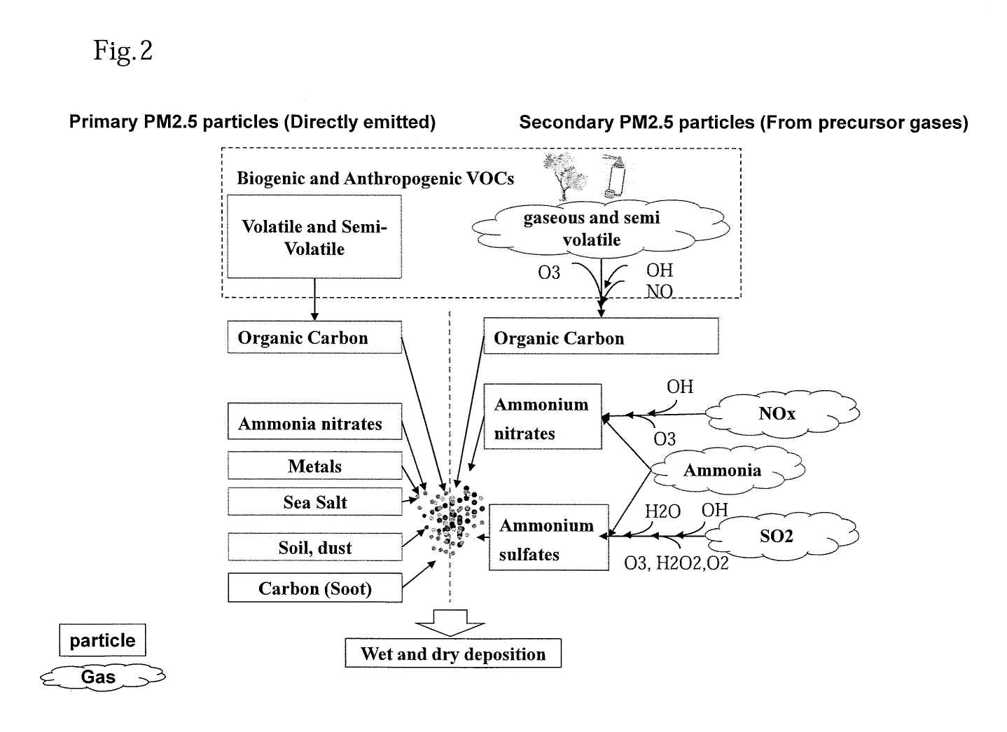

According to the literature, fine particulates PM2.5 are recognized as one of the air pollutants most likely to affect asthma. Primary PM2.5 are directly emitted into the atmosphere and includes suspended carbon (elemental carbon), metals, dust and sea salt. Secondary PM2.5 forms when precursor gases undergo chemical and physical transformations in the atmosphere, such as Organic Aerosols (OA) from Volatile Organic Compounds (VOCs), Ammonium Sulfate (NH4)2SO4 from Sulfur Dioxide (SO2) and ammonia (NH3) as well as Ammonium Nitrate (NH4NO3) from Nitrogen Oxides (NOx) and ammonia (NH3) as shown in FIG. 2.

Formation of secondary PM is often initiated by reaction of precursors with the OH radical sensitive to many of the same factors such as sunlight, NOx, and VOCs than ozone (O3) formation. Atmospheric aerosols, such as ammonium nitrate and some organic compounds, are semi-volatile and found in both gas and particle phases. Taking into account these precursors is important as it influences PM2.5 particles composition and therefore asthma effects.

The air pollutant measurement module preferably uses the same steps as presented in the method described in US 2013/01790178 A1 for greenhouse gases. The modules presented in the method described in US 2013/01790178 A1 are adapted to the measurement of fine particles PM2.5 and the air pollutant measurement module also adds three additional steps to deliver modeled hourly concentrations forecasts of fine particles PM2.5 and hourly concentrations forecasts of fine particles PM2.5 without the anthropogenic sources on the predetermined geographical area.

Observation Module

Instead of performing daily concentration measurements as indicated in the method described in US 2013/01790178 A1, the method preferably does hourly measurements of concentrations in a first plurality of locations distributed on the entire terrestrial globe and saves said hourly concentration measurements in an observation module.

Instead of performing daily flux measurements as indicated in the method described in US 2013/01790178 A1, the method preferably does hourly flux measurements in a first plurality of locations distributed on the entire terrestrial globe and saves said hourly concentration measurements in an observation module.

In particular, satellite observations of PM2.5 are performed using MODIS instrument located on Aqua and Terra satellites. In addition, in North America, continuous measurements of surface aerosol concentrations at hourly resolution are made available thanks to monitoring stations participating in the US EPA AIRNow program. Observations are available through a subscription service to AIRNow gateway and are processed with a small delay making them suitable for near real-time assimilation. There are over 600 sites measuring PM2.5.

As indicated in the method described in US 2013/01790178 A1, the method preferably does measurements of satellite parameters, meteorological parameters, marine parameters and ecosystem parameters in a third plurality of locations distributed on the terrestrial globe and saves said parameter measurements in the said observation module.

In particular, measurements of these parameters, preferably follow the observations of parameters described in the Rapid Refresh Model, http://rapidrefresh.noaa.gov/, from the National Oceanic and Atmospheric Administration with:

Upper-Air Observations

| | |
|---|---|
| Rawinsonde and dropwind sonde—NOAA | Velocity, Temperature, |
| Upper Air Observations | Relative Humidity |

Profiler and Radar Observations

| | |
|---|---|
| Profiler—NOAA Profiler Network | Velocity |
| Profiler—915 MHz—NOAA | Velocity, Virtual temperature |
| Profiler Network | |
| Radar—Velocity Azimuth Display | Velocity |
| (VAD)-WSR-88D—NOAA network | |
| Radar reflectivity—National Weather | Reflectivity |
| Service Enhanced Radar Image | |

AirCraft observations

| | |
|---|---|
| Aircraft (Aircraft Communications, | Velocity, Temperature |
| Addressing, and Reporting | |
| System—ACARS) | |
| Aircraft—Water Vapor Sensing | Relative Humidity |
| System, (Aircraft | |
| Communications, Addressing, and | |
| Reporting System—ACARS) | |

Surface observations

| | |
|---|---|
| Surface/METAR from the Aviation | Temperature, Dewpoint |
| Weather Center from NOAA, | temperature, Velocity, |
| | Visibility, Pressure, |
| | Cloud, Weather |

Ocean observations

| | |
|---|---|
| Buoys/ships—NOAA Ocean Climate | Temperature, sea level |
| Observation Program, | pressure |

Satellite observations

| | |
|---|---|
| NOAA geostationary satellite | Satellite derived winds, cloud- |
| GOES-15, and from polar | top pressure, temperature, |
| orbiting satellites NOAA 15, 18, 19 | moisture information |
| and European Metop A and B | |
| Advanced Microwave Sounding | Radiances |
| Unit (AMSU), Microwave | |
| humidity Sounder (MHS), High Resolution | |
| Infrared Radiation Sounder (HIRS) | |
| GPS—Precipitable water from NOAA | Precipitable water |
| WindSat scatterometer from NOAA, | Velocity |
| MODIS | Land use change, fires |

Lightening observations

| | |
|---|---|
| Lightning (proxy reflectivity) from | Cloud-to-ground lightning |
| National Lightning | flashes |
| Detection Network (NLDN) and the | |
| Vaisala Global Lightning | |
| Detection Network (GLD360) | |

As mentioned above, according to a preferred embodiment, the method preferably performs measurements as presented in the method described in US 2013/01790178 A1. Alternatively, the measurements can also be retrieved from data sources of observations providing such information. In such a case, one can understand, that measurements are not performed but only used or integrated.

Exchange Module

As indicated in the method described in US 2013/01790178 A1, the method performs a flux evolution modeling of the said air pollutant on the globe by means of an exchange module. Instead of starting from the Holocene as indicated in the method described in US 2013/01790178 A1, the modeling for the exchange module is done in the current year. The exchange module preferably does not include a fossil module since anthropogenic sources are preferably modeled in the current year in the ascending inventories module.

The exchange module preferably uses the same solar and energy modules as indicated in the method described in US 2013/01790178 A1. The measurement of radiances from the observation module enables one to validate the data of the solar module with real measurements. The measurement of radiances, pressure, moisture information, cloud, temperature and reflectivity are integrated into the energy module to validate its modeling and to refine its parameterization.

Instead of using MOM3 and CDIAC DB1012 for CO2 as indicated in the method described in US 2013/01790178 A1, the Ocean module preferably uses the MADE model with the SOA-VBS model for the sea salt emissions.

Instead of using JSBACH for CO2 as indicated in the method described in US 2013/01790178 A1, the Biosphere module preferably determines biogenic sources using the MEGAN model available at http://lar.wsu.edu/megan/, and dust emissions from the MADE model with the SOA-VBS model. The measurements by the satellite observations of land use change are integrated in the MEGAN model in order to validate the change in surface cover.

Instead of using GFEDv2 for CO2 as indicated in the method described in US 2013/01790178 A1, the fire module preferably determines fire emissions from MODIS satellite observed wildfires for PM2.5 as indicated by Giglio et al. 2003, “An Enhanced Contextual Fire Detection Algorithm for MODIS”.

Ascending Inventories Module

Instead of performing a weekly anthropogenic emission modeling as indicated in the method described in US 2013/01790178 A1, the method performs an hourly anthropogenic emissions modeling of said air pollutant by means of an ascending inventories module, said module integrating the raw data of emissions for a plurality of facilities. In particular, to model fine particles PM2.5 and its precursor emissions in the atmosphere such as Ammonia (NH3), Sulfur Dioxide (SO2), Nitrogen Oxides (NOx), VOCs, Elemental Carbon (EC), Metals, the process preferably uses specific anthropogenic sources. The ascending module preferably uses the U.S. EPA NEI-2011 emissions inventory available at http://www.epa.gov/ttnchie1/net/2011inventory.html instead of EDGAR 4.0 for CO2 as indicated in the method described in US 2013/01790178 A1. It notably includes point emissions (e.g. facilities), area, onroad and nonroad emissions.

Instead of using the seasonality indicated in the method described in US 2013/01790178 A1, the hourly emissions of 76 species on a defined EPA 4-km emission grid are given in this inventory. The Onroad and Nonraod emissions are preferably done through U.S. EPA NMIM processing http://www.epa.gov/otaq/models/nmim/420r05024.pdf. Daily and week-end emissions are generated to account for weekend emission changes preferably using the U.S. EPA “Technical Support Document (TSD) Preparation of Emissions Inventories for the Version 6.0. 2011 Emissions Modeling Platform”. Likewise, seasonal differences in the emissions are calculated from the NEI-2011.

Extraction Module

Instead of using the ECMWF, as indicated in the method described in US 2013/01790178 A1, the method extracts, by means of an extraction module, the weather forecast data from preferably the Rapid Refresh model (RAP), http://rapidrefresh.noaa.gov, and its associated nested High-Resolution Rapid Refresh (HRRR), http://ruc.noaa.gov/hrrr/, which has a 3 km grid spacing. Meteorological observations are used to validate their modeling and to refine their parameterization. The meteorological versions of the RAP and HRRR are providing forecasts each hour with an 18- and 15-hr forecast length, which will preferably extend in the future to days, weeks and months with increasing computer-processing capacity.

Transport Module

As indicated in the method described in US 2013/01790178 A1, the method performs using said flux evolution modeling, said hourly anthropogenic emissions modeling, and said weather forecast data, an atmospheric transport modeling of the said air pollutant by means of a transport module. In particular, in replacement of the TM5 atmospheric transport model, the method preferably uses the chemistry versions of the RAP and HRRR models, which are the RAP-Chem, http://ruc.noaa.gov/wrf/WG11_RT/, at 13 km grid spacing and the HRRR-Chem at 3 km grid spacing. They are based on WRF-Chem, which provides the capability to simulate trace gases and particulates such as sulphate-nitrate-ammonium-water aerosols interactively with the meteorological fields using several treatments for photochemistry and aerosols. This is indicated in Grell et al. 2005, “Fully coupled online chemistry within the WRF model” and Fast et al. 2006, “Evolution of ozone, particulates, and aerosol direct forcing in an urban area using a new fully-coupled meteorology, chemistry, and aerosol model”. The RAP-Chem includes sophisticated chemistry and aerosol modules for the formation of Organic Carbon from VOCs and for the deposition by including the parameterization of Ahmadov et al. 2012, “A volatility basis set model for summertime secondary organic aerosols over the eastern United States in 2006” with preferably the Regional Atmospheric Chemistry Mechanism RACM model. The RAP-Chem will provide chemical boundary conditions for the HRRR-Chem. The same RAP-Chem fractional break-up of aerosol species is used for the HRRR-Chem. The RAP-Chem provides 48 hours forecasts. The HRRR-Chem can deliver hourly PM2.5 concentrations out to 15 hours forecasts of the current day at 3-km resolution, which will preferably extend to days, weeks and months in the future with increasing computer-processing capacity. This online treatment of the physical and chemical processes accounts for a more accurate simulation of the transport for such high resolution simulations as indicated in Grell et al. 2011 “Integrated Modeling for Forecasting Weather and Air Quality: A Call for Fully Coupled Approaches” and Grell et al. 2004 “Online versus offline air quality modeling on cloud-resolving scales”. Additionally, aerosols affect the global climate directly by enhancing atmospheric reflectivity and indirectly by affecting the growth and reflectivity of clouds. Aerosol results are integrated in the energy module to refine its parameterization.

Data Inversion and Assimilation Module

As indicated in the method described in US 2013/01790178 A1, the method calculates the final fluxes of said air pollutant, by means of a data inversion and assimilation module using said fluxes modeling performed by the exchange module, said hourly anthropogenic emissions modeling performed by the ascending inventories module, said atmospheric transport modeling performed by the transport module and said measurements saved in said observation module. In replacement of the Green synthesis and ensemble Kalman Filters, the method preferably uses a 3D-variational Gridpoint Statistical Interpolation (GSI) as indicated in Wu et al. 2002 “Three-dimensional variational analysis with spatially inhomogeneous covariances”.

Weighting Module

As indicated in the method described in US 2013/01790178 A1, the method weights, by means of a weighting module, the said final fluxes so as to provide final weighted fluxes,

In particular, the weighting module uses the same principle based on the game theory with a macro-economic modeling of production activities of economic sectors (energy, industrial processes, product use, agriculture, land use, land use change and forestry, waste and other sources) of each state and its fossil energy use. Fine particles PM2.5 are regulated in the United States according to the U.S. EPA NAAQS and the cost of complying to air pollution standards induces the technological changes and therefore the reduction in emission levels. Each state has developed a specific State Implementation Plan with regulatory measures, http://www.epa.gov/airquality/urbanair/sipstatus/regionalpgs.html. Instead of using an emission markets model to account for the effects of regulatory and technological changes and the reduction of emission levels as indicated in the method described in US 2013/01790178 A1, the method preferably estimates costs of emission control technologies using the Control Strategy Tool (CoST) and the EPA Air Pollution Control Cost Manual from U.S. EPA, http://www.epa.gov/ttn/ecas/costmodels.html. It enables to develop control strategies that match control measures to emission sources using algorithms such as “Maximum Emissions Reduction” and “Least Cost”. Each control measure is determined from the regulations indicated in each State Implementation Plan.

Geocoding Module

Instead of calculating weekly emissions of said air pollutant of said geographical area as indicated in the method described in US 2013/01790178 A1, the method calculates, using said final weighted fluxes and said hourly anthropogenic emissions modeling performed by the ascending inventories module, the hourly emissions of said air pollutant of said geographical area, by means of a geocoding module using correcting coefficients and comprising at least one geographic information system.

Additional Steps to Deliver PM2.5 Hourly Concentrations

The air pollutant measurement module performs three additional steps to achieve the intended results compare to the method described in US 2013/01790178 A1.

As a first step, the method adjusts the said raw data of emissions of said air pollutant of the said ascending inventories module with the said hourly emissions of said air pollutant of said geographical area. The hourly emissions are preferably used to refine the emission inventories from the plurality of facilities in the ascending inventories module. Running the air pollutant measurement module with corrected inventories from facilities in the U.S. EPA NEI-2011 model enables to enhance the delivery of final concentrations forecasts by the transport module.

As a second step, the method extracts, from said transport module, the hourly concentrations forecasts of said air pollutant in said determined geographical area.

As a third step, to assess health effects in the geocoding module (block 500), background concentrations forecasts have also to be determined to assess the difference in exposure between modeled PM2.5 concentrations forecasts and the PM2.5 concentrations forecasts without the contribution of anthropogenic sources. This will enable to determine the total public health burden relative to “nonanthropogenic background” concentrations forecasts that would occur in the absence of anthropogenic emissions. The air pollutant measurement module (block 100) is preferably launched without the anthropogenic component from the ascending inventories module to simulate background levels in the absence of anthropogenic emissions. The PM2.5 hourly background concentrations forecasts are then extracted from the transport module on the continental United States at a 3 km resolution.

This continuous hourly measurement of air pollutants, the refinement of anthropogenic emissions and taking into account air pollutant, weather and climate interactions completes BENMAP to deliver hourly concentration forecasts as well as hourly background concentration forecasts each hour with lengths of hours, days, weeks and months of fine Particles PM2.5 in μg/m3 on a 3×3 km2 spatial resolution onto the U.S at a higher spatial and temporal resolution, in near-real time, in the future with forecasts lengths and with more accuracy.

2. Exposure Module

The goal of estimating population exposure forecasts is to provide the necessary input for concentration-response functions, so that the incidence module (block 300) can forecast adverse health effects. The method preferably uses BENMAP averaging functions to average hourly concentrations forecasts and determine averaged concentrations forecasts.

Averaged Concentrations Forecasts

The metrics used in concentration-response functions are the hourly, daily, weekly, monthly, quarterly and annual average of PM2.5. The exposure module (block 200) estimates the air pollution exposure for each grid-cell, with the assumption that people living within a particular grid-cell experience the same air pollution levels. It uses the PM2.5 modeled and background hourly concentrations forecasts from the air pollutant measurement module (block 100) and the following equation can be used to determine the daily average for a day j:

$$Dail y {average}_{j} = {\sum }_{i = 1}^{24} \frac{{PM}_{2.5 , i}}{24}$$

Where PM2.5,i are the hourly values at 3×3 km2 grid from the air pollutant measurement module (100). Daily air pollution data is preferably used, as there are for example variations between weekdays and weekends of air pollution. An average can be performed weekly of air pollutants. The data set consists of approximately 7 days×24 hr=168 realizations of PM2.5,i over a week such as:

$$Weekl y {average}_{j} = {\sum }_{i = 1}^{168} \frac{{PM}_{2.5 , i}}{168}$$

The quarterly average of PM2.5 can be defined as being the average of a quarter such as: January-March or April-June or July-September or October-December. The data set preferably consists of approximately 91 days×24 hr=2184 realizations as can be determined thereafter:

$$Quarterl y {average}_{j} = {\sum }_{i = 1}^{2184} \frac{{PM}_{2.5 , i}}{2184}$$

The annual average of PM2.5 can also be defined as being the average of four quarterly averages defined as: January-March, April-June, July-September, October-December

$$Annual {average}_{j} = {\sum }_{i = 1}^{4} \frac{Quarterly {Average}_{i}}{4}$$

The median PM2.5 average can also be determined as being the median of values throughout the year.

Population Forecasts

The exposure module (block 300) preferably uses the same principle than the BENMAP population module to determine populations forecasts and enhances it from a 12×12 km2 into a 3×3 km2 spatial resolution.

Assigning Block-Level Census 2010 into Race-Ethnicity-Gender-Age Groups

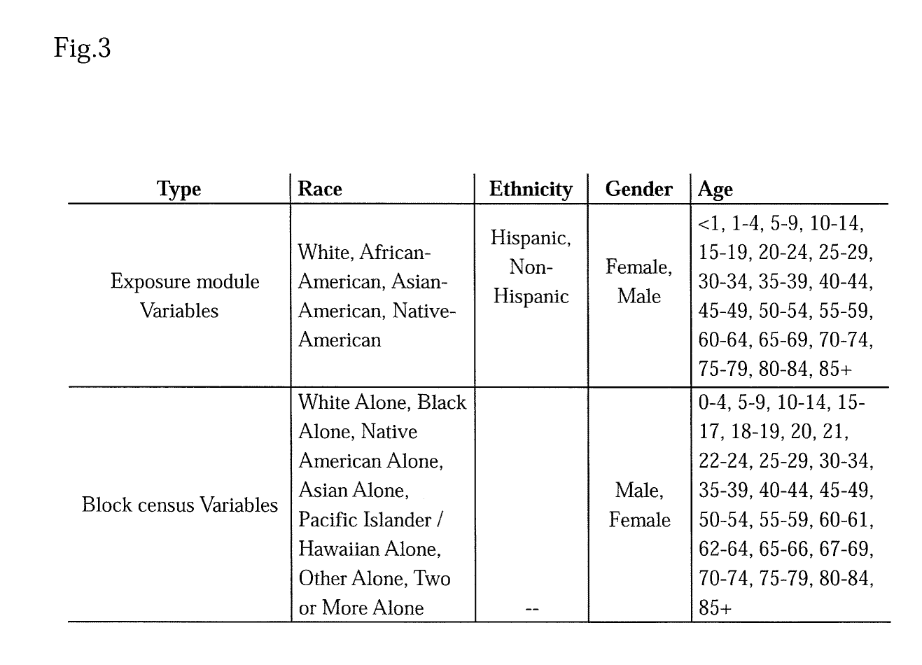

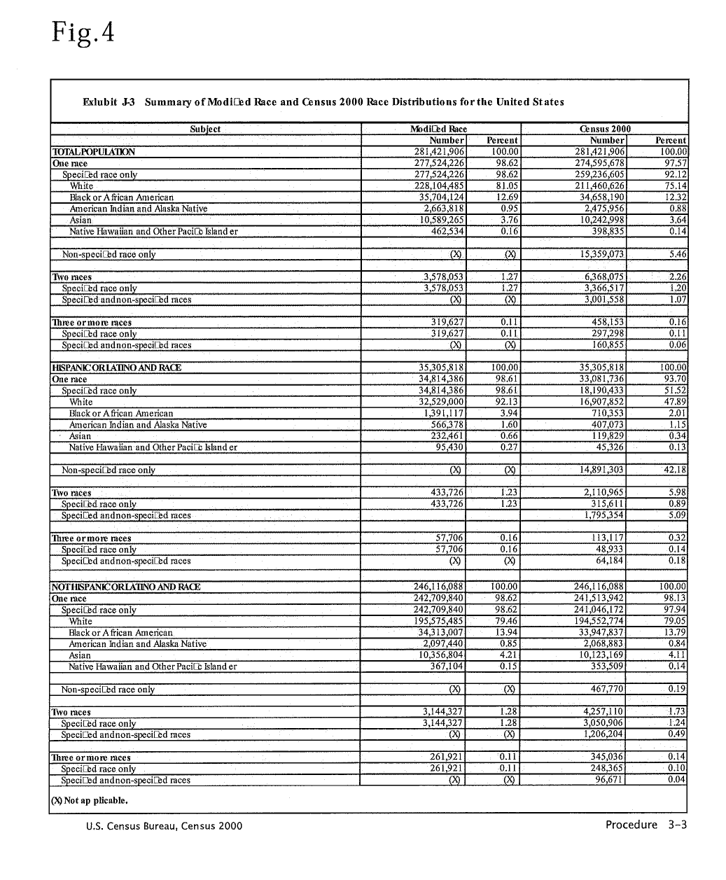

To best determine health effects according to epidemiological studies, the population dataset should have 304 unique race-ethnicity-gender-age groups: 4 racial groups, 2 ethnic groups, 19 age groups and 2 gender groups (4×2×19×2=304) as presented in FIG. 3. The term “population grid cell” refers to a cell within a grid definition and the foundation for calculating the population level is preferably the 2010 Census block data and tract-level Summary File 1 (SF1) available at http://www2.census.gov/census_2010/04-Summary_File_1/. There are about 5 million “blocks” in the United States, and for each block, there are 304 race-ethnicity-gender-age groups. The initial block file from the U.S. Census Bureau has 7 racial categories and 23 age groups, as opposed to the 4 and 19 needed in the exposure module as shown on FIG. 3. The following steps preferably converts the 2010 population data at block-census level into the correct 304 race-ethnicity-gender-age population groups:
1. Some age groups are combined in the block-level SF1 data to match the age groups wanted for the exposure module e.g. age groups 15-17 and 18-19 are combined to create the 15-19 age group. Then, in the case of the 0-4 age group, it is split into <1 and 1-4 using the tract-level SF1 data, which gives the fraction of 0-4 year-olds who are below 1.
2. The tract-level SF1 data is then used to calculate the fraction of Hispanics in each ethnically aggregated subpopulation from the block-level data, by age and sex. These fractions are used to distribute each age-sex-race-block-level datum into Hispanics and non-Hispanics.
3. The Census of Population and Housing: Modified Age/Race, Sex and Hispanic Origin Files (MARS) data files http://www.census.gov/popest/research/modified/MRSF2000.pdf preferably serves to reorganize the variables that come initially in the SF1 file into the correct race-ethnicity-gender-age groups. The “Other” race category is assigned in two steps. First, based on the national MARS data, it is estimated how many people in the “multi-racial” category checked off “some other race” as one of their races, for Hispanics and non-Hispanics separately. In each age-sex-race-block-level datum, those people are added to “other race” category to create the re-distribution pool, analogously to the method implemented by Census while creating MARS data in FIG. 4. Second, based on the national re-allocation fractions for Hispanics and non-Hispanics (derived from the MARS data), the “Other” race is assigned into the four races of interest and “multi-race”. After the assignment of the “Other” race category, “multi-racial” category is assigned to the four racial categories, using state fractions of these races in each age-sex-race-block level datum.

Assigning the Population into 3 km Grid-Cells

A population grid preferably integrates the Census block data into obtain a 3 km population grid cells following the HRRR atmospheric transport 3 km resolution from the air pollution measurement module (block 100). If the geographic center of a Census block falls within a population 3 km grid-cell, the block population is assigned to this particular population grid-cell. The population grid keeps track of the total number of people in each race-ethnic group by county within a particular population grid-cell. The exposure module assumes that all age-gender groups within a given race-ethnic group have the same geographic distribution.

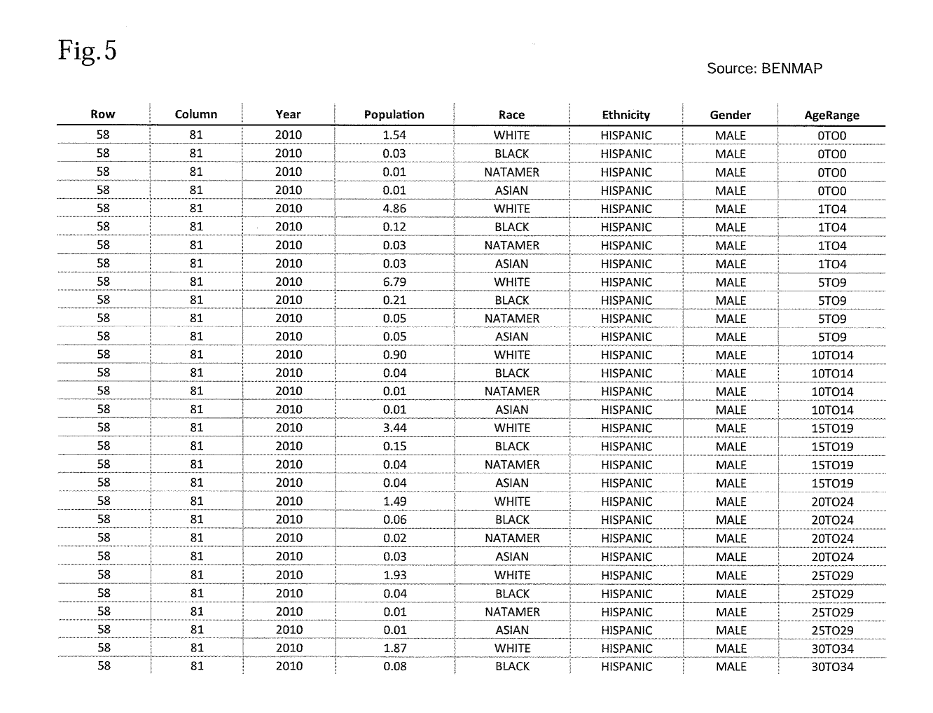

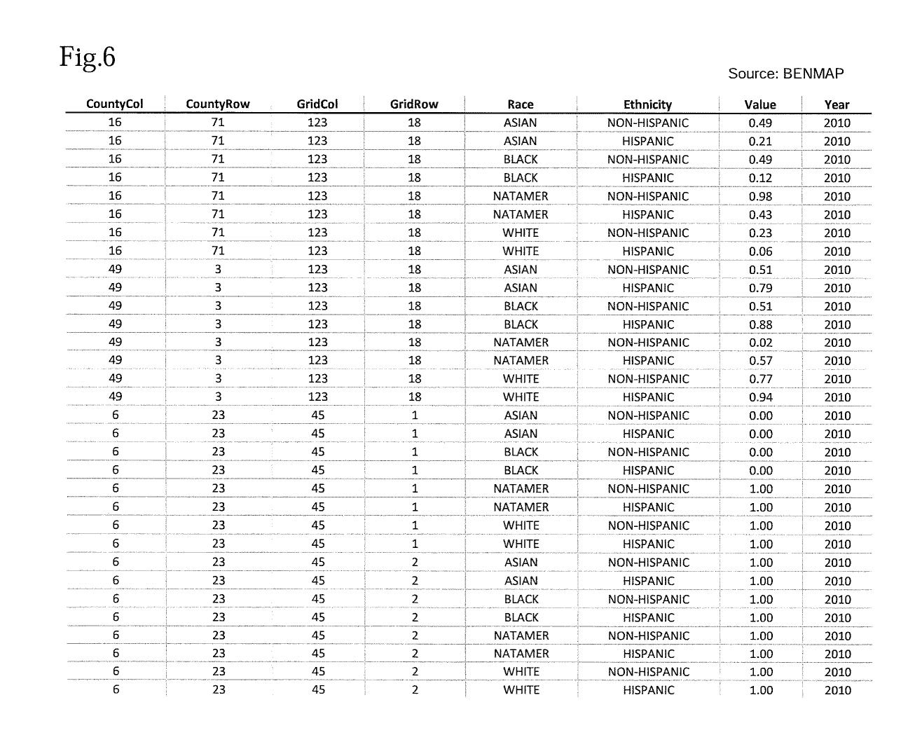

The gridding preferably generates two tables. One table in FIG. 5 has the number of people in each 3 km grid cell for each of the 304 race-ethnicity-gender-age demographic groups presents an example of the population file. The Row and Column uniquely identify each grid cell. The Race, Ethnicity, Gender and AgeRange variables are precisely defined. A second table in FIG. 6 tracks the fraction of the total population in each of the eight race-ethnic groups that comes from each county in the United States. The CountyCol and CountyRow uniquely identify each county, and the GridCol and GridRow uniquely identify each grid cell. The Value variable gives the fraction of the total population in the grid cell for a given race-ethnic group that comes from the “source” county. When a grid cell lies completely within a county, then the fraction is 1. When a grid cell is in more than one county, then the sum of the fractions across the counties for a given race-ethnic group is equal to 1. In FIG. 6 the grid cell (GridCol=123, GridRow=18) is the fraction of Asian Non-Hispanic coming from county (CountyCol=16, CountyRow=71) is 0.49 and for county (CountyCol=49, CountyRow=3) the fraction is 0.51. In this case, about half the population of Asian Non-Hispanics comes from each of the two counties. In the case of Black Hispanics, the fraction from county (CountyCol=16, CountyRow=71) is only 0.12, with most Black Hispanics in this grid cell e.g. 0.88 coming from county (CountyCol=49, CountyRow=3). The total number of people in a county is kept to thereafter forecast the population.

County-Level Forecasts

To determine county-level forecasts, the method preferably uses Woods & Poole (2012) http://www.woodsandpoole.com/pdfs/CED12.pdf county-level forecasts for each year from 2000 through 2040, by age and gender for non-Hispanic White, African-American, Asian-American, and Native-American and for all Hispanics. For each non-Hispanic subset of the population and each year from 2000-2040, the Woods and Poole population for that year is divided by the Woods and Poole population for that subset in 2010. The results serve as the growth coefficients for the non-Hispanic subsets of each race. A similar calculation is used to determine the growth rates for the Hispanic population. It is assumed that each Hispanic race grows at the same rate, and use these growth rates for the Hispanic subsets of each race.
1. The 86 age groups from Woods and Poole are aggregated to match the 19 used in the exposure module.
2. The county geographic boundaries used by Woods and Poole are more aggregated than the county definitions used in the 2010 Census and those in the exposure module. The Federal Information Processing Standards (FIPS) codes used by Woods and Poole are not always the standard codes used in the Census. To make the Woods and Poole data consistent with the county definitions in the exposure module, the Woods and Poole data is disaggregated and some FIPS codes are changed to match the U.S. Census as done in BENMAP.
3. Growth Ratios with Zero Population in 2000 are calculated. There are a small number of cases where the 2010 county population for a specific demographic group is zero, so the ratio of any future year to the year 2010 data is undefined. In these relatively rare cases, statewide and national totals are prepared and ratios are used at the higher levels of geographic aggregation as done in BENMAP.

The county-level data are county-level ratios of a “future” year (2000-2040) for each county and each of the 304 race-ethnicity-gender-age groups.

To calculate the population forecast for the upcoming years in age groups for an epidemiological study that may include a portion of one of the pre-specified demographic groups in FIG. 3, it is preferably assumed that the population is uniformly distributed in the age group. For example, the number of children ages 3 through 12 is calculated as follows:

age₃₋₁₂=½age₁₋₄+age₅₋₉+⅗age₁₀₋₁₄

Woods & Poole provides the county-level population forecasts used to calculate the scaling ratios. To estimate population levels for the years after the last Census in 2010, the 2010 Census-based estimate is scaled with the ratio of the county-level forecast for the future year of interest over the 2010 county-level population level. The forecasting of a single population variable such as children ages 4 to 9 where the gth population grid-cell is wholly located within a given county for the year 2015 can be calculated as:

$${age}_{4 - 9 , g , 2015} = {age}_{4 - 9 , g , 2010} \cdot \frac{{age}_{4 - 9 , country 2015}}{{age}_{4 - 9 , country 2010}}$$

In the case, where the gth grid-cell includes “n” counties in its boundary. The module preferably estimates the fraction of individuals in a given age group (e.g., ages 4 to 9) that reside in the part of each county within the gth grid-cell. Then, the module can calculate this fraction by simply dividing the population all ages of a given county within the gth grid-cell by the total population in the gth grid-cell:

$$fraction of {age}_{4 - 9 , g in {county}_{c}} = \frac{{age}_{all , g in {county}_{c}}}{{age}_{4 - 9 , g}}$$

Multiplying this fraction with the number of individuals ages 4 to 9 in the year 2010 gives an estimate of the number of individuals ages 4 to 9 that reside in the fraction of the county within the gth grid-cell in the year 2010:

age_{4-9,g in country}_{_{c}}_{,2010}=age_{4-9,g,2010}·fraction of age_{4-9,g in country}_{_{c}},

To then forecast the population in 2015, the module preferably scales the 2010 estimate with the ratio of the county projection for 2015 to the county projection for 2010:

$${age}_{4 - 9 , g in {county}_{c ,} 2015} = {age}_{4 - 9 , g in {county}_{c} , 2010} \cdot \frac{{age}_{4 - 9 , {county}_{c} , 2015}}{{age}_{4 - 9 , {county}_{c} , 2010}}$$

Combining all these steps for “n” counties within the gth grid-cell, the population of persons ages 4 to 9 in the year 2015 is forecasted as follows:

$${age}_{4 - 9 , g , 2015 } = {\sum }_{c = 1}^{n} {age}_{4 - 9 , g , 2010} \cdot \frac{total {pop}_{g in {county}_{c}}}{total {pop}_{g}} , \frac{{age}_{4 - 9 , {county}_{c} , 2015}}{{age}_{4 - 9 , {county}_{c} , 2010}}$$

In the case where there are multiple age groups and multiple counties, the module first calculates the forecasted population level for individual age groups, and then combines the forecasted age groups. In calculating the number of children ages 4 to 12:

$${age}_{4 - 9 , g , 2015} = {\sum }_{c = 1}^{n} {age}_{4 - 9 , g , 2010} \cdot \frac{total {pop}_{g in {county}_{c}}}{total {pop}_{g}} , \frac{{age}_{4 - 9 , {county}_{c} , 2015}}{{age}_{4 - 9 , {county}_{c} , 2010}}$$

$${age}_{10 - 14 , g , 2015} = {\sum }_{c = 1}^{n} {age}_{10 - 14 , g , 2010} \cdot \frac{total {pop}_{g in {county}_{c}}}{total {pop}_{g}} , \frac{{age}_{4 - 9 , {county}_{c} , 2015}}{{age}_{4 - 9 , {county}_{c} , 2010}}$$

$${age}_{4 - 12 , g , 2015} = {age}_{4 - 9 , g , 2015} + \frac{3}{5} {age}_{10 - 14 , g , 2015}$$

Since the Woods and Poole projections only extend through 2040, existing projections and constant growth factors can be used to provide additional projections. To estimate population levels beyond 2040, the module linearly extrapolates from the final two years of data. For example, to forecast population in 2045, the module can calculate it as follows:

age_{4-9,2045}=age_{4-9,2040}+5(age_{4-9,2040}−age_{4-9,2039})

To determine population exposure forecasts, the exposure module (block 200) preferably delivers hourly, daily, weekly, quarterly and annual averages of modeled and background air pollutant concentration forecasts, consistent with latest observations of fine particle levels (PM2.5) in μg/m³ and a 3×3 km² resolution for the past year and the upcoming hours, days, weeks, months, quarters and year based on forecasts lengths. It also preferably delivers the corresponding population forecast of 304 groups in the current and future years of interest for each corresponding 3×3 km2 grid-cell onto the U.S.

3. Incidence Module

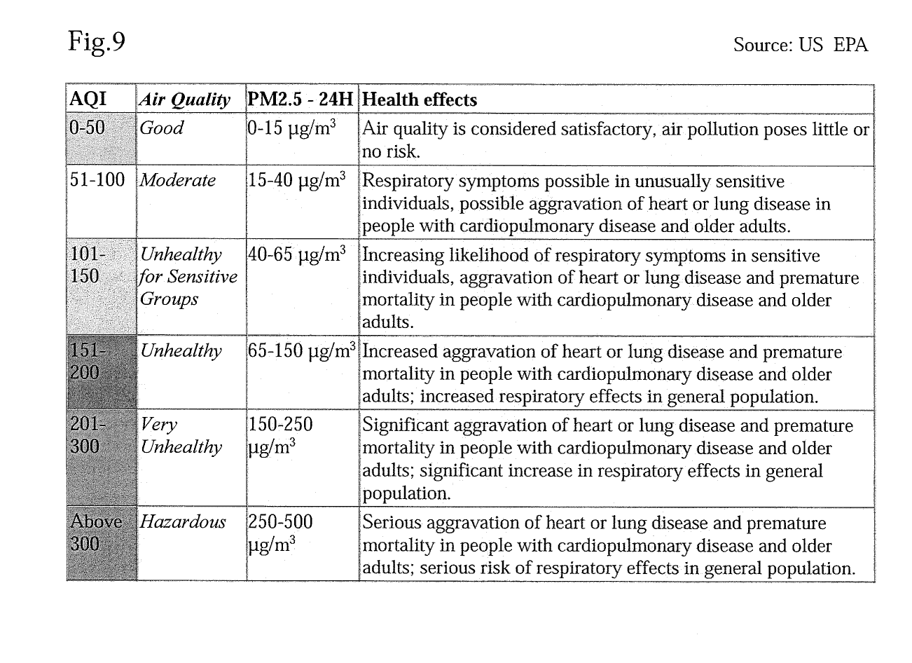

In addition, the method completes BENMAP by determining near real-time and future Air Quality Index from averaged concentration forecasts according to forecast lengths from the exposure module (Block 200). The averaged concentrations forecasts can be converted into the Air Quality Index (AQI) and health effects as indicated on FIG. 9 by U.S. EPA www.epa.gov/ttn/oarpg/t1/memoranda/rg701.pdf. Decisions about pollutant concentrations at which to set the various AQI breakpoints that delineate the various AQI categories for each pollutant specific sub-index within the AQI draw directly from the underlying health information and epidemiological studies that supports the NAAQS review http://www.epa.gov/ttn/naaqs/standards/pm/s_pm_index.html.

To determine averaged forecasted air quality health effects, the incidence module (block 300) can use the same principle than the incidence model of BenMAP. Health impact functions usually estimate the percent change in an adverse health effect associated with a given pollutant change. To estimate the absolute change in incidence using these functions, the baseline incidence rate of the adverse health effect, or the number of cases experienced by a given population per unit of time needs to be determined. Baseline incidence rates are commonly based on study or research estimates and the same principle can be applied universally to any new research used to define the baseline incidence rate of a disease.

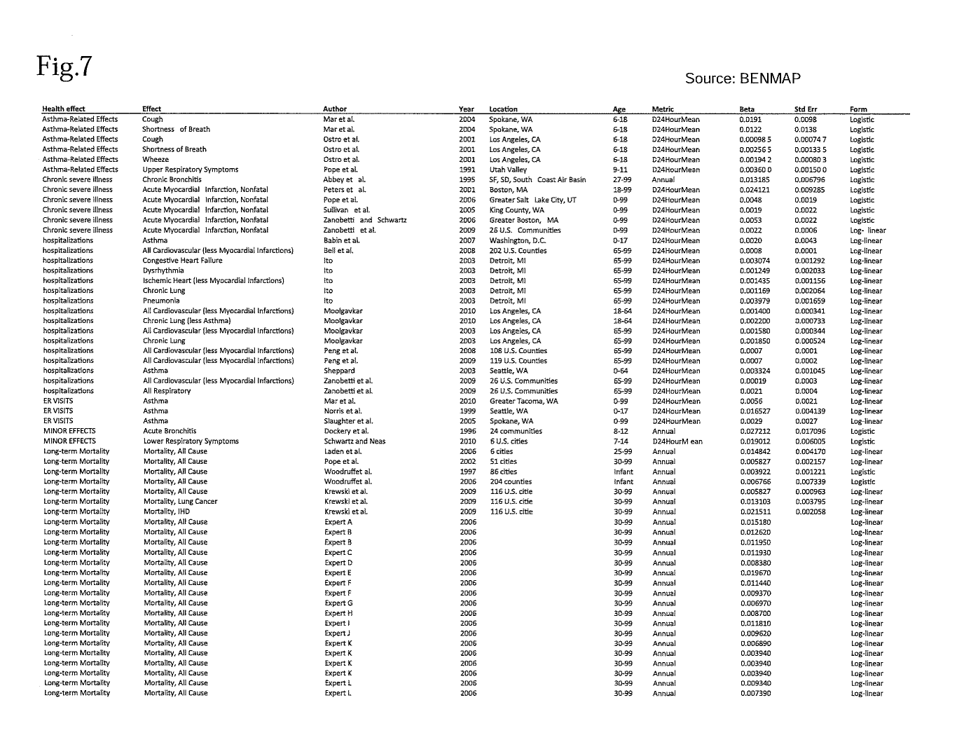

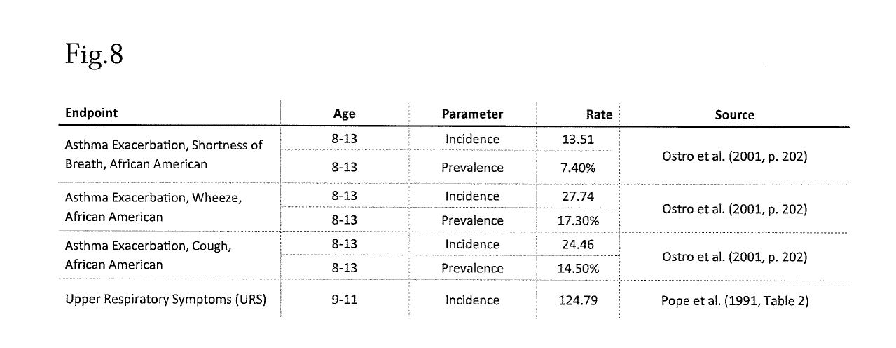

In this method, where the focus is on asthma, Mar et al. (2004) in FIG. 7 studied the effects of various size fractions of particulate matter on respiratory symptoms of adults and children with asthma, monitored over many months. The study was conducted in Spokane, Wash., a semiarid city with diverse sources of particulate matter. Data on respiratory symptoms and medication use were recorded daily by the study's subjects, while air pollution data was collected by the local air agency and Washington State University. Subjects in the study consisted of 16 adults the majority of whom participated for over a year and nine children, all of whom were studied for over eight months. Among the children, the authors found a strong association between cough symptoms and several metrics of particulate matter, including PM2.5. However, the authors found no association between respiratory symptoms and PM of any metric in adults. Mar et al. therefore concluded that the discrepancy in results between children and adults was due either to the way in which air quality was monitored, or a greater sensitivity of children than adults to increased levels of PM air pollution. The study reported results for population ages 7-12. For comparability to other studies, the results can be applied to the population of ages 6 to 18. Mar et al. (2004) did not report the incidence rate for each type of asthma exacerbation. The daily cough rate per person from Ostro et al. (2001, p. 202) is applied here e.g. of 0.145 as shown in FIG. 8. The incidence module preferably delivers the Asthma Exacerbation, Cough incidence rate to the population aged from 6 to 18 years old on a 3×3 km2 over the entire U.S.

Once having determined the incidence rate of the health effect, the incidence module enables one to determine the health effects on a 3×3 km2 basis over the entire U.S. for the population of interest using the incidence rate, the modeled averaged and background averaged concentration forecasts variation and the population forecasts from the exposure module (block 200). Following the same study, from Mar et al. (2004), the functional form used is the logistic one, with a coefficient β of 0.01906 and a standard error σ_{β} of 0.00983 as shown in FIG. 7. Hence, according to the logistic model:

$$Δ Health = Δ y . pop = ( y - {y}_{o} ) \cdot pop = - ( 1 - \frac{1}{( 1 - {y}_{o} {e}^{β ( {PM}_{o} - {PM}_{c} )} + {y}_{o}} ) \cdot {y}_{o} \cdot pop$$

The lower and upper bound of the coefficient (β) and its standard error (σ_{β}) can be determined as:

β_{lower bound}=β−(1.96·σ_{β})=0.01906−(1.96*0.00983)=−1.1×10⁻⁴

β_{upper bound}=β+(1.96·σ_{β})=0.01906+(1.96*0.00983)=3.8×10⁻²

By indicating the incidence rate and assuming that there is a daily average of 10 μg/m3 concentration forecast increase in PM2.5 delivered by the exposure module (block 200) when comparing the modeled averaged vs. the background averaged concentrations forecast on a given 3 km grid cell, one can determine the health effects within the bounds suggested by the two estimates as follows:

$$Δ {y}_{1} = ( {y}_{1} - {y}_{o} ) = ( \frac{1}{( 1 - 0.145 ) {e}^{- 1.1 \times {10}^{- 4} \times - 10} + 0.145} - 1 ) \cdot 0.145 = - 1.3 \times {10}^{- 4}$$

$${Δy}_{2} = ( {y}_{2} - {y}_{o} ) = ( \frac{1}{( 1 - 0.145 ) {e}^{3.8 \times {10}^{- 2} \times - 10} + 0.145} - 1 ) \cdot 0.145 = 5.4 \times {10}^{- 2}$$

By multiplying with the corresponding population aged 6-18 in the 3 km grid-cell, one can determine the corresponding range of health effects. Since, the daily rate of new cough is used, the value can be multiplied by 100 to get the incidence rate per 100 people aged 6-18 years old.

ΔHealth_{asthma exacerbations,cough,pop 6-18(1)}=1.3×10⁻⁴·pop_{pop 6-18}=−0.01

ΔHealth_{asthma exacerbations,cough,pop 6-18(2)}=−5.4×10−2·pop_{pop 6-18}=5.4

The result for the population is an estimated health impact increase between 0 and 5 asthma exacerbations, cough per day on the grid cell for the general population aged 6 to 18 years old per 100 persons. The health effects can be extrapolated over the entire U.S by cumulating the health effects over the period of interest on each grid-cell by using the average concentration forecasts, the averaged background concentrations forecasts, the corresponding population forecasts and the incidence data. The incidence module delivers forecasted air quality health effects on the period of interest over hours, days, weeks, months, quarters and years in the past and the future according to forecasts lengths for each grid cell at a 3×3 km2 for the entire U.S.

4. Geocoding Module

The results of the incidence module (Block 300) are transferred to the geocoding module (block 400) comprising a GIS coordinate system (Geographic Information System) enabling one to geocode the results and notably, the hourly concentrations forecasts, the hourly background concentrations forecasts (Block 100), the averaged modeled concentrations forecasts, the averaged background concentrations forecasts, the population forecasts (Block 200), the Air Quality Index, the incidence data, the forecasted air quality health effects (Block 300). The geocoding module delivers on a GIS map the forecasted air quality health effects of the population of interest on an hourly, daily weekly, monthly, quarterly and annual timeframe in the past and the future according to forecasts lengths.

II. Other Air Pollutant Health Effects

The same process is used to determine the other health effects of fine particles PM2.5 as presented on FIG. 7 and for any new research indicating the incidence of a disease from an air pollutant, the principle is universal.

III. System

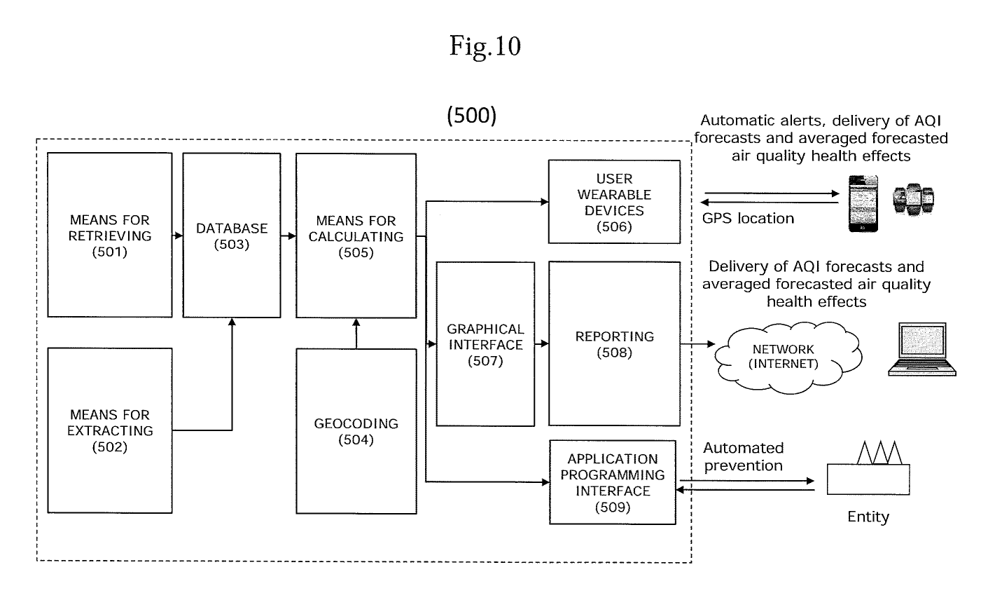

According to the invention, the method described above is implemented by means of a data processing system (FIG. 10, Block 500) comprising means for retrieving air pollutants (Block 501), at least one centralized database (Block 503) comprising the air pollutant measurement module, means for extracting (Block 502), comprising means for transferring automated data, and also ensuring the necessary interface with the communication networks. The system according to the invention also comprises means for calculating (Block 505) such as a plurality of dedicated information servers, computers, mainframes, etc. and means for geocoding (Block 504). The system comprises, one or more interfaces for wearable devices (Block 506), one or more graphical interfaces (Block 507), in addition means for reporting (Block 508), and one more application programming interfaces (Block 509). As has been said above, each of the modules of the process can advantageously be implemented in the form of software, hardware or a combination of both. In addition, given the relative complexity of the process according to the invention, it is clear that the system, which implements it requires strong computing power, important data storage capacity as well as reliable and fast means for communicating.

As stated above, the invention therefore aims to provide an efficient health prevention of air pollutants for a given geographical area, and does this by executing the method according to the invention. This efficient prevention can either constitute the final result intended to be directly used on a wearable device such as a mobile application or in the prevention management system of an entity or taken into consideration by individual or institutional users.

A centralized server receives the hourly concentrations forecasts in μg/m³ from the air pollutant module (Block 100) as well as the averaged hourly, daily, weekly, monthly, quarterly, and annual modeled and background concentrations forecasts, the population forecasts (Block 200), the Air Quality Index, the incidence data, the forecasted air quality health effects (Block 300) at a 3×3 km² spatial resolution onto the U.S. in the past and in the future according to forecasts lengths.

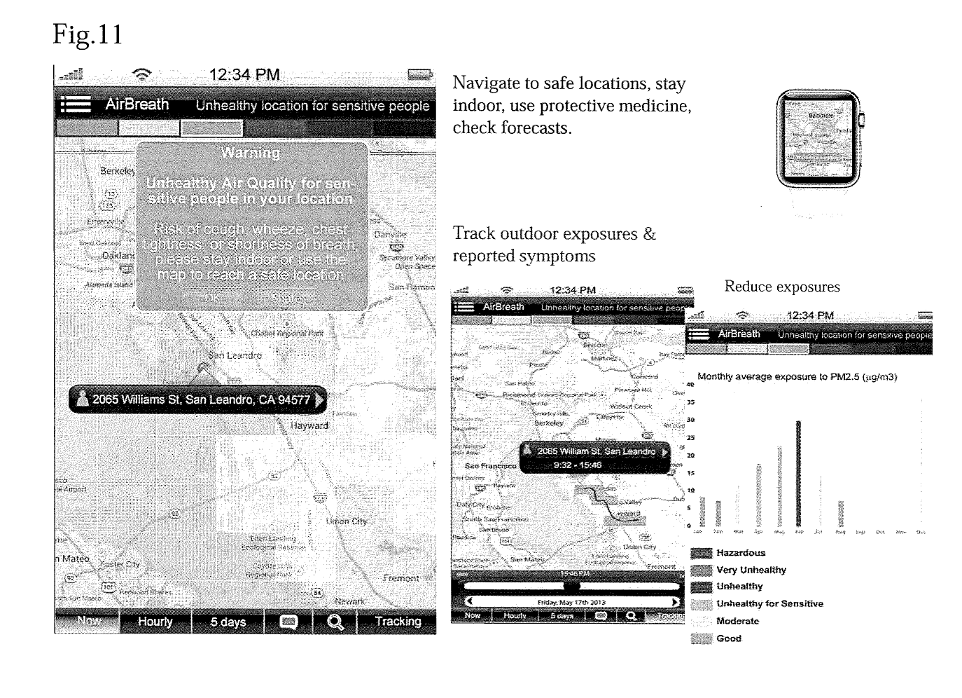

In the first case, air pollutants lead to major health effects, which individuals cannot avoid since information is provided at a very coarse level. There is today no personalized information or variability forecast to help an individual manage his life accordingly. To enable prevention, users are equipped with a wearable device (block 506) such as a mobile phone e.g. a smart phone or a traditional cellular phone for example where the mobile application is installed. The application can also be installed in any wearable devices such as smart watches, tablets or bracelets as show in FIG. 11, which can track a user's location. The central server receives continuously the GPS locations of users to geoposition them and when a user is located in a grid-cell with a harmful Air Quality Index (AQI) defined from the concentration forecast, an alert is automatically triggered and sent via notification or SMS to inform the user of the harmful air pollutant levels in his or her location. The system automatically alerts users when exposures are unhealthy for them and when one opens the application, the AQI map centered on his or her GPS location corresponding to his or her local area is loaded. The application pulls the air pollution data to show the personalized air pollution level in the grid-cell as well as the air pollution levels in the vicinity to find clean air areas and safer places to move as shown in FIG. 11. Maps are overlaid on a web mapping service offering for example satellite imagery, street maps, and street view perspectives for ease of use. As air pollution data is forecasted hourly in near real-time with forecasts lengths, this allows users to change behavior by for example changing location, staying indoor or using preventive medicine. It also enables a user to plan activities ahead such as waiting to go to a certain place until the outdoor air improves. Instead of continuously downloading maps on the mobile app, this automatic alerting principle provides AQI data with accuracy and high resolution while preserving battery and bandwidth. An interactive questionnaire can also be triggered when a user changes grid-cell from his GPS positioning to request feedback for example if the user felt symptoms, changed behavior and is feeling better and therefore assess the system efficiency. The GPS location of users is monitored on the central server and compared with the air quality map to quantify the average outdoor exposure over time and space. This enables to also track user exposures over time as shown in FIG. 11 and present it to the user as an incentive to reduce exposures.

The averaged hourly, daily, weekly, monthly, quarterly, and annual health effects in the past and the future from the incidence module (block 300) are also positioned geographically on the GIS map of the wearable device. If a user has a specific pathology such as a respiratory or a cardiovascular problem, the user can preferably indicate exposure variable such as race, ethnicity, gender, age as shown on FIG. 3, along with the pathology, as shown on FIG. 7, on the application and receive a specific personalized alert regarding that pathology on the wearable device as shown on FIG. 122. When located in an area where the pathology is higher than the incidence data or another threshold of interest, the alert is automatically triggered for that pathology. Users who would not indicate a specific pathology would receive alerts for all pathologies. Associating diseases and geographies enables to determine the attractiveness of a location as a function of a users' pathology in the past and the future. The health effects are shown on an hourly, daily, weekly, monthly, quarterly and annual temporal scale with a map to enable user to plan long term behavior change and show safer locations to live, work, go to school and plan outdoor activities depending on pathologies.

The benefit of the system from the invention is to use high spatial resolution (3×3 km2), real-time and future forecasted air quality health effects to automatically and very precisely prevent health risks on the short, medium and long term by triggering a behavior change in users. It also enables to efficiently personalize health risks according to user pathologies. Hence, the wearable device using hourly and forecasted AQI along with past and future averaged health effects determined from the invention will empower the general population and sensitive individuals to reduce exposures and efficiently enhance health prevention.

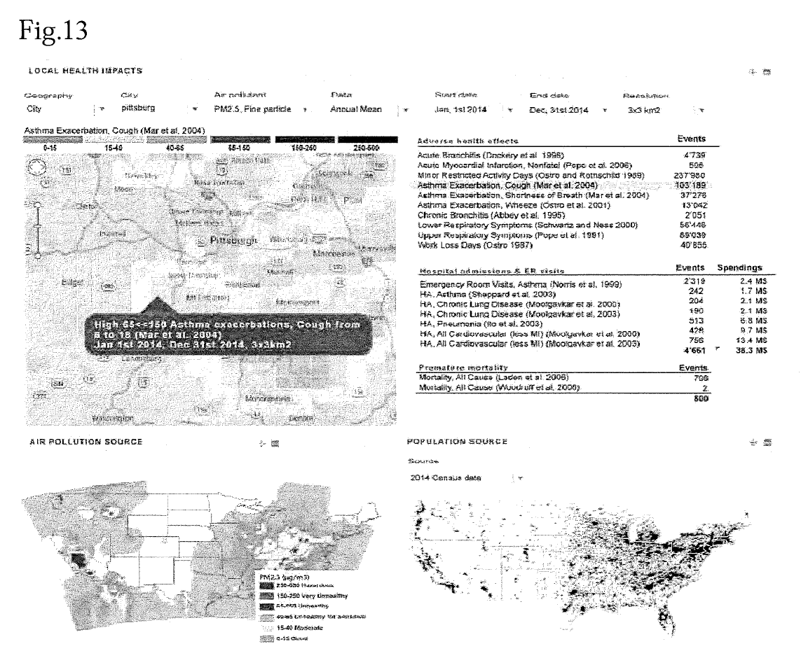

In a second case, the wearable device is connected to a centralized Internet platform accessible by Internet to users equipped with a personal computer or similar connected equipment, and this, preferably with a secured access via a graphical interface. This interface enables users to navigate on the map throughout these grids by scaling them with a web mapping service offering for example satellite imagery, street maps, and street view perspectives. The access rights to data are allocated as a function of user profiles and can be limited geographically to preserve the confidentiality of information (Block 508). The results are continuously transmitted, preferably in real time, to this Internet platform. Users of the system can advantageously put in place several axes of analysis including, but not limited to the fields of results of the air pollutant measurement module, the exposure module, the incidence module and the geocoding module to perform detailed analyses. Users can search locations, coordinates (latitude, longitude), view and analyze the types of air pollutants, values of fluxes, hourly concentrations forecasts, timeframe, uncertainty, AQI forecasts, averaged hourly, daily, weekly, monthly, quarterly and annual concentrations forecasts of an air pollutant, population (race, gender, ethnicity, age), health incidence, hourly, daily, weekly, monthly, quarterly and annual forecasted air quality health effects of a plurality of given geographical areas covering the entire globe in the past and the future years as shown on FIG. 13. They can also view their personal exposures as well as exposure reduction over time. Lower income populations, usually more exposed, which are alerted via SMS on a cell phone can assess on the online platform clean air locations, then change their behavior and prevent exposures. Reporting can be performed as a function of the desired geographical area (world, continents, continental regions, states, countries, regions, counties, grid-cells), the desired time period (hour, day, week, month, year), the types of pollutants and the type of health effects. The user then selects the desired area and the system aggregates the sum of the health effects in the area and the time period considered. Health effects reports, intended for institutional users, can be generated at any time. They preferably include the air pollutant, population, health incidence and health effects.

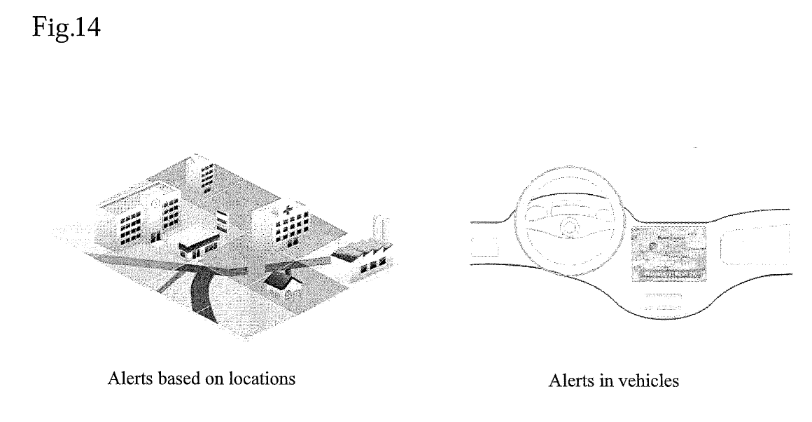

In a third case, the data is integrated with an Application Programming Interface (API) (block 509) into the prevention management system of an entity. The system can especially be integrated within the prevention management system of healthcare focused institutional entities, currently limited in their knowledge of exposures such as healthcare providers, payers, health agencies and policy-makers to target preventive actions. For example, the API can transfer real-time and forecasted AQI and averaged forecasted health effects in the past and the future into their prevention management system to locate the communities at risk based on their address in their databases and automate phone calls to alert them as shown on FIG. 14. When their home is for example under high pollution levels at a given time of the day, an automatic alert is sent into the prevention management system and a healthcare operator can call them to advise preventive measures or an automatic email is sent to the users. Another possibility is to setup more long term preventive actions such as identifying suitable locations to build new homes or helping to relocate sensitive populations at risk such as individuals with underlying respiratory and cardiovascular diseases, children, elderly and pregnant women when located in areas at risk based on the projected health effects data. They will project accurately the local health impacts and potentially identify new locations to live for populations at risk depending on their pathologies. Programs to share specific prevention best practices e.g. asthma management can be targeted to specific locations or protective medicine such as inhalers can be distributed in selective locations at risk for asthma individuals for example. The system can help these professionals to better understand what triggers or exacerbates effects to develop better population level prevention and also enhance advocacy on high-risk areas to reduce air pollution levels. This is simple and efficient and does not exist to current knowledge. It can also be used by industrial facilities to assess locations at risk and projected health impacts with air pollutant concentrations increase when setting up a new plant.

The API can also be interfaced with the data management software of pharmaceutical companies. The geopositioned averaged health effects indicate sensitive locations where to target drugs where populations at risk are located and more likely to need treatments. They can also identify points of distribution such as pharmacies in sensitive areas. The API can also be integrated into vehicles to enable individuals driving to avoid air pollution and sensitive locations. In particular, triggering automatic alerts to taxis and medical vehicles such as ambulances could help protect passengers by avoiding these locations, closing windows or using air filters. The API can also be integrated into the housing information system of an entity to create a housing air quality index for sensitive populations to assess the suitability of locations to live.

What is claimed is:

1. A method for determining at least one forecasted air quality health effect caused in a determined geographical area by at least one air pollutant, said method being performed by a system comprising at least one computing device, which comprises one or more processors and one or more computer-readable storage media operatively coupled to at least one of the processors, wherein said method includes:
a step of retrieving, by means of an air pollutant measurement module, hourly concentrations forecasts of said at least one air pollutant;
a step of determining, by means of an exposure module, population exposure forecasts using said hourly concentrations forecasts and population related data;
a step of deriving, by means of an incidence module, said at least one forecasted air quality health effect caused by said at least one air pollutant using said population exposure forecasts;
a step of localizing, by means of a first geocoding module comprising at least one geographic information system, said at least one forecasted air quality health effect caused by said at least one air pollutant so as to assess said at least one forecasted air quality health effect in said determined geographical area.

2. The method of claim 1, wherein the surface of said determined geographical area is comprised between 1 km2 and 10,000 km2.

3. The method of claim 1, wherein said at least one health effect is selected from the group consisting of asthma, acute bronchitis, acute myocardial infarction, cardiovascular, respiratory, chronic bronchitis, chronic lung, congestive heart failure, cough, dysrhythmia, ischemic heart, lower respiratory symptoms, mortality from all cause, mortality from ischemic heart disease, mortality from lung cancer, pneumonia, shortness of breath, upper respiratory symptoms and wheeze.

4. The method of claim 1, wherein said step of retrieving hourly concentrations forecasts of said at least one air pollutant comprises the following steps:
use hourly concentration measurements of said at least one air pollutant measured in a first plurality of locations distributed on the entire terrestrial globe and save said hourly concentration measurements in an observation module;
use hourly flux measurements of said at least one air pollutant measured in a second plurality of locations distributed on the entire globe and save said hourly flux measurements in said observation module;
use measurements of satellite parameters, meteorological parameters, marine parameters and ecosystem parameters measured in a third plurality of locations distributed on the terrestrial globe and save said satellite parameters, said meteorological parameters, said marine parameters and said ecosystem parameters in said observation module;
extract, by means of an extraction module, weather forecast data from at least one data source;
perform a flux evolution modeling of said at least one air pollutant on the globe by means of an exchange module modeling the natural and anthropogenic sources and sinks;
perform an hourly anthropogenic emissions modeling of said at least one air pollutant by means of an ascending inventories module, said ascending inventories module integrating the raw data of emissions for a plurality of facilities;
perform, using said flux evolution modeling, said hourly anthropogenic emissions modeling and said weather forecast data, an atmospheric transport forecast of said air pollutant by means of a transport module;
calculate final fluxes of said at least one air pollutant, by means of a data inversion and assimilation module, by making use of said flux evolution modeling, said hourly anthropogenic emissions modeling, said atmospheric transport forecast and said measurements saved in said observation module;
weight, by means of a weighting module, said final fluxes so as to provide final weighted fluxes;
calculate, using said final weighted fluxes and said hourly anthropogenic emissions modeling, the hourly emissions of said at least one air pollutant of said geographical area, by means of a second geocoding module comprising at least one geographic information system; and
adjust said raw data of emissions for a plurality of facilities by making use of said hourly emissions of said at least one air pollutant of said geographical area; and
retrieve said hourly concentrations forecasts of said at least one air pollutant from the said atmospheric transport forecast.

5. The method of claim 4, wherein said transport module makes use of the RAP-Chem model and the HRRR-Chem model.

6. The method of claim 1, wherein said population related data includes a block-level census population data, a population grid, a county level forecast and a population forecast.

7. The method of claim 1, wherein said incidence module uses health incidence data, concentration response functions and concentration response parameters from epidemiological studies and medical research used by BenMAP as well as the Air Quality Index issued by U.S. EPA.

8. A system for determining at least one forecasted air quality health effect caused in a determined geographical area by at least one air pollutant, said system comprising at least one computing device which comprises one or more processors, one or more computer-readable storage media operatively coupled to at least one of the processors, and implementing an air pollutant measurement module, an exposure module, an incidence module and at least one geocoding module comprising at least one geographic information system,
wherein said system performs a method for determining at least one forecasted air quality health effect caused in a determined geographical area by at least one air pollutant, said method including the following steps:
a step of retrieving hourly concentrations forecasts of said at least one air pollutant, wherein said step of retrieving is performed by means of said air pollutant measurement module;
a step of determining population exposure forecasts of said at least one air pollutant using said hourly concentrations forecasts and population related data, wherein said step of determining is performed by means of said exposure module;
a step of deriving said at least one forecasted air quality health effect caused by said air pollutant using said population exposure forecasts, wherein said step of deriving is performed by means of said incidence module; and
a step of localizing said at least one forecasted air quality health effect caused by said air pollutant so as to assess said at least one forecasted air quality health effect in said determined geographical area, wherein said step of localizing is performed by means of said geocoding module.

9. The system of claim 8, wherein said at least one forecasted air quality health effect is determined on an hourly basis.

10. The system of claim 8, wherein said at least one forecasted air quality health effect is determined on a daily basis.

11. The system of claim 8, wherein said at least one forecasted air quality health effect is determined on a weekly basis.

12. The system of claim 8, wherein said at least one forecasted air quality health effect is determined on a monthly basis.

13. The system of claim 8, wherein said at least one forecasted air quality health effect is determined on a quarterly basis.

14. The system of claim 8, wherein said at least one forecasted air quality health effect is determined on an annual basis.

15. The system according to claim 8, further including an interfacing module for interfacing with at least one software application executed by a wearable device.

16. The system of claim 15, wherein said wearable device is a smartphone.

17. The system of claim 8, further comprising an online platform comprising at least one server hosting at least one website.

18. The system of claim 8, further comprising a framework allowing said system to be linked to a remote sanitary prevention management system.
